(19) United States
(12) Patent Application Publication
(10) Pub. No.: US 2020/0008983 A1
(43) Pub. Date: Jan. 9, 2020

(54) ADJUSTABLE LOOP SHAPED BANDAGE

(71) Applicant: John Linstrom, Polson, MT (US)

(72) Inventor: John Linstrom, Polson, MT (US)

(21) Appl. No.: 16/029,823

(22) Filed: Jul. 9, 2018

(51) Int. Cl. A61F 13/10; A61F 13/00; A61F 13/06

(52) U.S. Cl. CPC A61F 13/105; A61F 13/00063; A61F 2013/00817; A61F 2013/00578; A61F 2013/00655; A61F 13/068

(57) ABSTRACT

A bandage includes a first end portion having a first perimeter size and a bottom surface and a top surface. The bandage further includes a second end portion arranged opposite the first end portion, the second end portion having a second perimeter size with a bottom surface and a top surface. The second perimeter size may be smaller, larger or the same as the first perimeter size. An elongated portion extends between the first end portion and the second end portion. The first end portion is configured to be applied to a wound with the bottom surface of the first end portion contacting the wound, the elongated portion being configured to wrap around a body part positioned adjacent to the wound, and the bottom surface of the second end portion is configured to be secured to or near the top surface of the first end portion.

### TECHNICAL FIELD

The present disclosure relates generally to bandages, and more particularly to bandages for appendages.

### BACKGROUND

There are many existing types of bandages to cover various areas of the body. There are standard bandages for every day scrapes on more streamlined areas of the body such as an arm, leg, or torso. Of the standard bandages, there are mini-type bandages that are smaller and butterfly bandages for keeping cuts closed. There are also hourglass-shaped bandages for fingertips and H-shaped bandages for knuckles. However, these bandages do not always stay on the person. Furthermore, typical bandages are not designed for heavy wear and tear. For example, some bandages may peel off if worker applies the bandage and then keeps working. If a bandage is on a foot, the bandage may rub and wear off as the person walks around. Therefore, a sturdier bandage design is needed.

### SUMMARY

In one embodiment, a bandage is described. The bandage includes a first end portion having a first perimeter size and a bottom surface and a top surface. The bandage further includes a second end portion arranged opposite the first end portion, the second end portion having a second perimeter size with a bottom surface and a top surface. The second perimeter size is larger than the first perimeter size. An elongated portion extends between the first end portion and the second end portion. The first end portion is configured to be applied to a wound with the bottom surface of the first end portion contacting the wound, the elongated portion being configured to wrap around a body part positioned adjacent to the wound, and the bottom surface of the second end portion is configured to be secured to the top surface of the first end portion.

In some embodiments, the elongated portion comprises a flexible, deformable material. A dressing may be coupled to the first end portion. The dressing may include one of an antibiotic or antiseptic. The first end portion and second end portion may each comprise an adhesive on the bottom surface. The second end portion may completely cover the first end portion when the second end portion is secured to the top surface of the first end portion. In some embodiments, the body part may be a digit of a person. The body part may be a person. The middle portion may be free of adhesive.

In another embodiment, a method of applying a bandage to a person is described. The method includes adhering a bottom surface of a first part of a bandage to a person at a first location and wrapping a middle portion of the bandage around a body part of the person. The method also includes adhering an end of the bandage on a top surface of the first part of the bandage, the first part being completely encompassed by the end of the bandage.

In some embodiments, the middle portion may be configured to deform as it wraps around the body part. The body part may be a finger. The body part may be a toe. The body part may be a limb. In some embodiments, the method may include placing a dressing of the bandage over a wound of the body part, the dressing being located on the first end. In some instances, the dressing may include one of an antibiotic or antiseptic.

In another embodiment, a wound dressing is described. A contoured wound-covering end is configured to mount to a skin surface. A contoured connecting end is arranged opposite the wound-covering end and configured to connect to the wound-covering end or mount to the skin surface adjacent to the wound-covering end. A deformable elongated portion separates the wound covering end and the contoured connecting end. The deformable elongated portion is configured to wrap around a digit of a person.

In some embodiments, the wound-covering end may comprise a non-adhesive dressing. The non-adhesive dressing may include one of an antibiotic or antiseptic. In some instances, the adhesive end may completely cover the wound-covering end when the wound-covering end is applied to a wound.

### BRIEF DESCRIPTION OF THE DRAWINGS

The accompanying drawings and figures illustrate a number of exemplary embodiments and are part of the specification. Together with the present description, these drawings demonstrate and explain various principles of this disclosure. A further understanding of the nature and advantages of the present invention may be realized by reference to the following drawings. In the appended figures, similar components or features may have the same reference label.

FIG. 1 is a top-down view of an exemplary bandage in accordance with the present disclosure;

FIG. 2 is a bottom-up view of an exemplary bandage in accordance with the present disclosure 2;

FIG. 3 is a side view of an exemplary bandage in accordance with the present disclosure; and

FIG. 4 is an exemplary bandage attached to a person in accordance with the present disclosure.

While the embodiments described herein are susceptible to various modifications and alternative forms, specific embodiments have been shown by way of example in the drawings and will be described in detail herein. However, the exemplary embodiments described herein are not intended to be limited to the forms disclosed. Rather, the instant disclosure covers all modifications, equivalents, and alternatives falling within the scope of the appended claims.

### DETAILED DESCRIPTION

For as long as there have been wounds, there have been bandages. Bandages to stop the bleeding, bandages to put an antiseptic or other medication on, bandages to prevent diseases. However, in recent times, advancement in bandages has slowed. Bandage configurations have stagnated resulting in very little advancement or improvements in the design and functionality of bandages. For example, attempting to place and retain a bandage on a joint remains a difficult task. Bandages often fall of or peel off as a person continues about their daily life. The bandages themselves cannot withstand the rugged wear and tear of a joint covering. However, if the joint is used to the advantage of the bandage then it no longer becomes a detriment and turns into an asset.

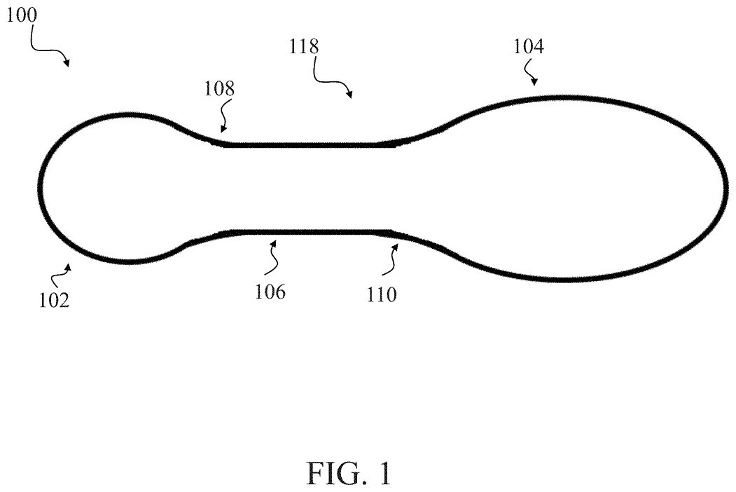

FIG. 1 is a top-down view of an exemplary bandage 100 in accordance with the present disclosure. A top side 118 of the bandage 100 is visible in FIG. 1. The bandage 100 may include a first end 102 and a second end 104 opposite the first end 102. An elongated portion 106 may extend between the first end 102 and second end 104.

The bandage 100 may comprise coated paper, plastic, or other flexible material. In some embodiments, the bandage 100 may comprise a fabric or plastic such as polyethylene or polyurethane, and in some embodiments, a latex strip or the like. The bandage 100 may be waterproof itself or may provide a watertight or airtight covering of a wound.

In some embodiments, the first end 102 may have a first size and shape. For example, in the embodiment shown, the first end is a smaller, circular shape having at least one contoured surface. The shape and size of the first end 102 may vary depending on size of the overall bandage. For example, the first end 102 may be placed on top of a wound, cut, scrape, or other injury. The elongated portion 106 may wrap around a digit or other body part. The second end 104 may then be placed either on top of the first end 102 or adjacent to the first end 102 such that the bandage 100 is firmly secured on a person's body.

In some embodiments, the second end 104 may be of a shape and size that is larger than the first end 102. In other examples, the second end 104 may have a size that this the same or smaller than the first end 102. In one example, the second end 104 may cover the first end 102 and therefore, to adequately adhere to a person's skin, the second end 104 may be an oval, circular, or other shape, and a perimeter of the shape that includes a contour and/or may be larger than a perimeter of a shape of the first end 102.

The first end 102 and second end 104 may transition to the middle, elongated portion 106. For example, a transition area 108 may be a slow, curved or contoured section between the first end 102 and the elongated portion 106. This may enable the bandage 100 to be flexible between the first end 102 and the middle portion 106. Similarly, a second transition area 110 may be a gradual curved section between the second end 104 and the elongated portion 106. This may enable the bandage 100 to be flexible between the second end 104 and the middle portion 106. For example, the gradual curved section may enable the bandage to smoothly adhere to a person without sharp edged. The transition area 108 may enable the elongated portion 106 to expand or stretch more when the bandage 100 is applied to a user.

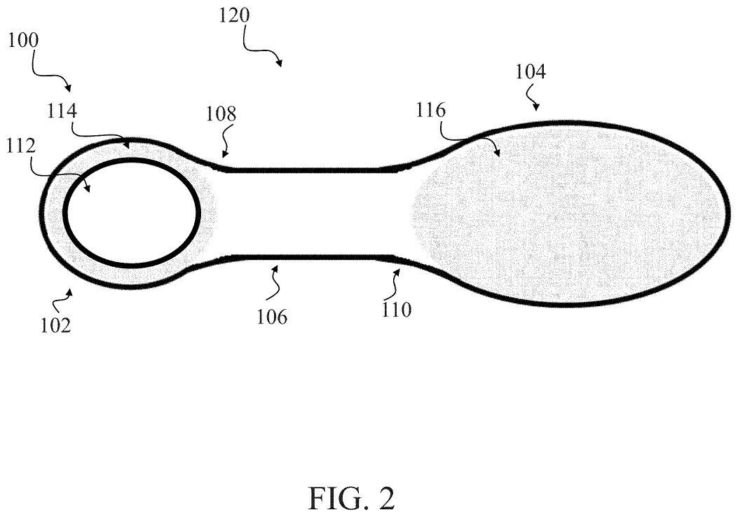

FIG. 2 shows a bottom side 120 of the bandage 100 described in FIG. 1. The bandage 100 shows the first end 102 and the second end 104 opposite the first end 102. The transition area 108 couples the first end 102 to the elongated middle portion 106. The second transition area 110 couples the elongated, middle portion 106 to the second end 104.

The first end 102 may include a dressing 112 on the bottom side 120 of the bandage 100. The dressing 112 may comprise, for example, gauze, non-adherent pad, non-adherent wet dressing, foam dressing, calcium alginate, hydrogel dressing, transparent dressing, alcohol preps, or the like. The dressing 112 may include antibiotic or antiseptic or the like.

The dressing 112 may be surrounded by an adhesive 114. The adhesive 114 may comprise any adhesive used in standard bandage applications. The second end 104 is also covered with an adhesive 116. The adhesive 114, 116 may enable the bandage 100 to couple or adhere to a user's skin. The adhesive may include, for example, an acrylate, methacrylate, epoxy diacrylate, vinyl resin, or the like. In other embodiments, the amount, shape or arrangement of the adhesives 114, 116 may be different from those shown in the figures, including an option of having no adhesive on one or both of the ends 102, 104.

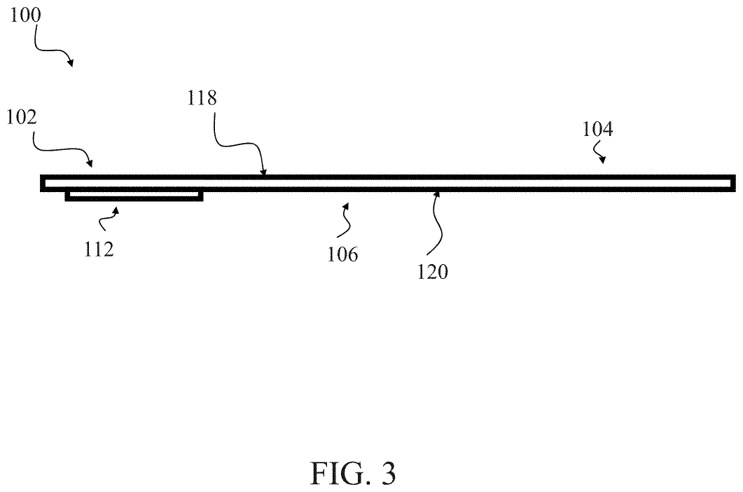

FIG. 3 is a side-view of the bandage 100. The bandage has a top-side 118 and a bottom-side 120 opposite the top-side 102. The first end 102 is opposite the second end 104 with the elongated portion 106 separating the two. The dressing 112 is shown on the bottom-side 120 here. The top and bottom sides 118, 120 are shown for mere orientation and the sides 120, 118 could be reversed.

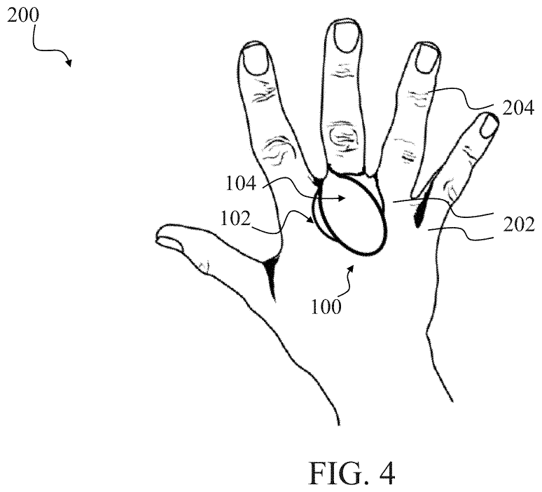

FIG. 4 is a view of the bandage 100 applied to a person's hand 200. The bandage 100 could also be applied to a person's foot or other flexible joint. As can be seen here, the first end 102 of the bandage 100 is applied over a wound (not shown) atop or proximate a person's knuckle 202. The elongated portion 106 of the bandage 100 is then wrapped around a person's digit 204. In some embodiments, the elongated portion 106 may be stretched or flexed or changed in length of the portion 106 as it is wrapped around the digit 204. The second end 104 of the bandage 100 may then overlap completely or cover a portion of the first end 102 to secure the bandage 100 to the user's hand 200. In some embodiments, the second end 104 may not cover or touch the first end 102 of the bandage 100 and may instead be placed next to or proximate the first end 102 of the bandage 100.

The bandage 100 may be configured to mount to any surface of a person. For example, the bandage 100 may be configured to be mounted to the outer or top side of the knuckle 202 and hand 200 as shown in FIG. 4, or may be configured to be mounted to an opposite side of the knuckle 202 along the palm or lower side of the hand 200. In some embodiments, the bandage 100 may be mounted to the palm surface or another surface of the hand 200 besides one of the knuckles 202. In yet further embodiments, one or more of the ends 102, 104 of the bandage 100 may be mounted to other limb, appendages, or other body parts of a person, such as around an ear, nose, forearm, lower leg, ankle, foot, or neck. As mentioned above, the ends 102, 104 may be at least partially directly connected to each other on a skin surface of the person, or may be mounted to the skin surface at spaced apart or directly adjacent positions on the skin surface.

Unless otherwise explained, any technical terms used herein have the same meaning as commonly understood by one of ordinary skill in the. The singular terms “a”, “an”, and “the” include plural referents unless the context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described herein. The term “comprises” means “includes.” All publications, patent applications, patents, and other references mentioned herein are incorporated by reference in their entirety for all purposes. The materials, methods, and examples are illustrative only and not intended to be limiting.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms such as “above,” “below,” “upper,” “lower,” or other like terms to describe a spatial relationship between various components or to describe the spatial orientation of aspects of such components should be understood to describe a relative relationship between the components or a spatial orientation of aspects of such components, respectively, as the device described herein may be oriented in any desired direction.

Various inventions have been described herein with reference to certain specific embodiments and examples. However, they will be recognized by those skilled in the art that many variations are possible without departing from the scope and spirit of the inventions disclosed herein, in that those inventions set forth in the claims below are intended to cover all variations and modifications of the inventions disclosed without departing from the spirit of the inventions. The terms “including” and “having” as used in the specification and claims shall have the same meaning as the term “comprising.”

What is claimed is:

1. A bandage comprising:
a first end portion having a first perimeter size, a bottom surface and a top surface;
a second end portion arranged opposite the first end portion, the second end portion having a second perimeter size, a bottom surface and a top surface;
an elongated portion extending between the first end portion and the second end portion;
wherein the first end portion is configured to be applied to a wound with the bottom surface of the first end portion contacting the wound, the elongated portion being configured to wrap around a body part positioned adjacent to the wound, and the bottom surface of the second end portion is configured to be secured to the top surface of the first end portion.

2. The bandage of claim 1, wherein the elongated portion comprises a flexible, deformable material.

3. The bandage of claim 1, further comprising:
a dressing coupled to the first end portion.

4. The bandage of claim 3, wherein the dressing includes one of an antibiotic or antiseptic.

5. The bandage of claim 1, wherein the first end portion and second end portion each comprise an adhesive on the bottom surface.

6. The bandage of claim 1, wherein the second end portion completely covers the first end portion when the second end portion is secured to the top surface of the first end portion.

7. The bandage of claim 1, wherein the body part comprises a digit of a person.

8. The bandage of claim 1, wherein the second perimeter size is the same or smaller than the first perimeter size

9. The bandage of claim 5, wherein the elongate portion is free of adhesive.

10. A method of applying a bandage to a person, the method comprising:
adhering a bottom surface of a first part of a bandage to a person at a first location;
wrapping a middle portion of the bandage around a body part of the person;
adhering an end of the bandage on a top surface of the first part of the bandage, the first part being completely encompassed by the end of the bandage.

11. The method of claim 10, wherein the middle portion is configured to deform as it wraps around the body part.

12. The method of claim 10, wherein the body part is a finger.

13. The method of claim 10, wherein the body part is a toe.

14. The method of claim 10, wherein the body part is a limb.

15. The method of claim 10, further including:
placing a dressing of the bandage over a wound of the body part, the dressing being located on the end.

16. The method of claim 15, wherein the dressing includes one of an antibiotic or antiseptic.

17. A wound dressing comprising:
a contoured wound-covering end configured to mount to a skin surface;
an contoured connecting end arranged opposite the wound-covering end and configured to connect to the wound-covering end or mount to the skin surface adjacent to the wound-covering end;
a deformable elongated portion separating the wound covering end and the contoured connecting end;
wherein the deformable elongated portion is configured to wrap around a digit of a person.

18. The wound dressing of claim 17, wherein the wound-covering end comprises a non-adhesive dressing.

19. The wound dressing of claim 18, wherein the non-adhesive dressing includes one of an antibiotic or antiseptic.

20. The wound dressing of claim 17, wherein the contoured connecting end completely covers the wound-covering end when the wound-covering end is applied to a wound.
